(19) United States
(12) Patent Application Publication
(10) Pub. No.: US 2015/0335268 A1
(43) Pub. Date: Nov. 26, 2015

(54) Measuring Breathing of a Patient During a Magnetic Resonance Examination

(71) Applicants: Stephan Biber, Erlangen (DE); Andreas Fackelmeier, Thalmassing (DE); Robert Rehner, Neunkirchen am Brand (DE)

(72) Inventors: Stephan Biber, Erlangen (DE); Andreas Fackelmeier, Thalmassing (DE); Robert Rehner, Neunkirchen am Brand (DE)

(21) Appl. No.: 14/709,546

(22) Filed: May 12, 2015

(30) Foreign Application Priority Data
May 20, 2014 (DE) 102014209488.7

(51) Int. Cl. A61B 5/08; A61B 5/00; A61B 5/053; A61B 5/055

(52) U.S. Cl. CPC A61B 5/0806; A61B 5/0555; A61B 5/7285; A61B 5/6892; A61B 5/0816; A61B 5/0535; A61B 5/0809; A61B 5/721

(57) ABSTRACT

A method is provided for measuring the breathing of a patient during a magnetic resonance examination with a magnetic resonance device, wherein the reflection properties of at least one coil element arranged beneath the patient are measured and evaluated to determine the breathing data that describes the respiratory situation at various times.

### CROSS-REFERENCE TO RELATED APPLICATIONS

This application claims the benefit of DE 10 2014 209 488.7, filed on May 20, 2014, which is hereby incorporated by reference in its entirety.

### TECHNICAL FIELD

The embodiments relate to a method for measuring the breathing of a patient during a magnetic resonance examination, to a measuring arrangement, and to a magnetic resonance device.

### BACKGROUND

Nowadays magnetic resonance imaging is to be regarded as being established, in particular in the medical sector. In this process, a patient is exposed to a strong magnetic field, the basic magnetic field or Bo field, so the patient's spins align. The aligned spins may then be excited by a high-frequency excitation and the decay of this excitation is measured as the magnetic resonance signal. Gradient fields are used to achieve a local resolution.

One possible source of errors or cause of artifacts in magnetic resonance examinations is the movement of a patient. Since magnetic resonance images require a certain amount of time, it is important that the patient lies still for the entire run time of the magnetic resonance examination. This assumption is not possible in the case of measurements in the region of the abdomen/thorax since the tissue to be imaged is, in any case, subject to movements due to the breathing of the patient. Various methods have already been developed to correct acquired magnetic resonance data with respect to the breathing of the patient, which is measured accordingly. It has likewise been proposed that breathing data of the patient be used to trigger a measurement. If magnetic resonance data and breathing data are acquired simultaneously, the breathing data may also be evaluated to subsequently sort the magnetic resonance data according to breathing phases (e.g., retrospective gating).

Some options have already been proposed for obtaining the breathing data that describes the breathing profile of the patient. It is known by way of example to carry out special magnetic resonance measurements, in particular, what are known as navigators. A one-dimensional measurement may be recorded in this connection in the region of a contrast transition, a delimitation between different tissues therefore, so a conclusion may be drawn about the breathing using the displacement. A delimitation of the diaphragm is conventionally measured in current navigators. The problem with using navigators is their time-consuming nature. There is also a greater electromagnetic exposure of the patient due to the additional magnetic resonance measurements, wherein the SAR increases. That may be limited, however, and this may lead to restrictions in the overall examination.

Alternatively, additional measuring devices for breathing have been proposed, in particular, breathing belts and breathing cushions. Such measuring devices are tightened around the stomach or chest of the patient or wedged between an anterior local coil and the stomach/chest of the patient. Increased, additional effort is required for correct arrangement and preparation of the breathing belt/cushion. Furthermore, external devices also have to be used in addition to the magnetic resonance device.

Further methods for measuring the breathing of a patient during magnetic resonance examinations are known from the ISMRM-Abstracts “An Alternative Concept of Non-sequence-interfering, Contact-free-Respiration Monitoring”, Proc. ISMRM 17 (2009) 753, and “An Alternative Concept of Non-Sequence-interfering Patient Respiration Monitoring”, Proc. ISMRM 16 (2008) 202. Therein, it is proposed to detect the respiratory movement by observing the reflection parameters of a high-frequency coil arrangement (e.g., body coil (BC)) that surrounds the entire patient in the manner of a cylinder. Other methods that deal with the observation of transmission parameters between an anterior local coil and a coil element behind the paneling of the patient-receiving mechanism of the magnetic resonance device are known from the subsequently published German patent application DE 10 2013 212 276.4, which is hereby incorporated by reference in its entirety.

### SUMMARY AND DESCRIPTION

The scope of the present invention is defined solely by the appended claims and is not affected to any degree by the statements within this summary. The present embodiments may obviate one or more of the drawbacks or limitations in the related art.

The embodiments are based on disclosing a method for measuring the breathing of a patient during a magnetic resonance examination that is (1) as simple as possible to implement, (2) robust, and (3) reliable.

To achieve this object, a method for measuring the breathing of a patient during a magnetic resonance examination is provided, which is distinguished in that the reflection properties of at least one coil element arranged beneath the patient are measured and evaluated to determine breathing data that describes the respiratory situation at various times.

The approach therefore includes the placement of a fixed coil element posteriorly to the patient in the region of the thorax/abdomen of the patient. The coil element may be arranged as close to the patient as possible in this connection, e.g., less than 10 cm or less than 5 cm from the body of the patient. If a transmission signal is emitted via this antenna and the returning signal is measured as a measuring signal, the characteristics thereof depend on the electromagnetic properties of the patient in the region of the abdomen/thorax, which are changed by the breathing, however. A conclusion may be drawn about the respiratory movement therefore by changing the high-frequency properties of the coil element. For the measurement of the electromagnetic effect advantage is taken of the fact that the magnetic field, which is emitted by the coil element, couples to the human body, and spreads far into the body. For example, if the patient breathes in, the lungs expand and the distribution of the tissue inside the sensitivity range of the coil element changes, so the signal reflected in the coil element, which acts as an antenna, also changes.

Test measurements have shown that the breathing cycles and holding of breath that occur may clearly be seen in this way. The amplitude and/or the phase of the returning signal from the coil element by way of example may be evaluated in the process. Very low transmitting powers are already sufficient in this connection, so it may be provided by way of example that a transmitting power of the coil element of less than 10 dBm (decibel-milliwatts) is used for measurement of the reflection properties. By way of example −10 dBm or −40 dBm may be used. The transmitting power is therefore considerably below a milliwatt, and this results in an insignificant exposure for the patient and therefore does not have a noteworthy effect on the SAR.

It may be emphasized in this connection that the method described here is sensitive to a change in the tissue distribution and not just to the expansion of the body in the anterior direction. This stands in contrast to methods that are sensitive to the change in the spacing of the surface of the body from a sensor located further away, by way of example when considering the transmissions between a coil positioned anteriorly on the surface of the patient and a coil element of a body coil arranged behind the cladding of the patient-receiving mechanism of the magnetic resonance device. The method is therefore much more suitable for (e.g., sick) patients whose breathing is very shallow and whose thorax or stomach expand only slightly. The posteriorly arranged coil element is located in the region in which the patient is lying, (e.g., on a patient couch), so therefore no movement of the coil element occurs there. However, it has nevertheless been found that the measurable changes in the tissue distribution during breathing supply a sufficiently clear signal due to the proximity to the patient.

It has also been found once the coil element is arranged posteriorly to the patient in the region of the abdomen or thorax that interference, which may occur due to arm movements, is reduced since the arms are shielded by the dielectric properties of the body itself, (e.g., of the torso), and this would not be the case with a coil arranged by way of example behind the cladding of a patient-receiving mechanism.

The use of a coil element arranged posteriorly, e.g., beneath the patient, close to the patient in the region of the abdomen- and/or thorax for measuring the breathing has a large number of advantages in particular also with respect to the subsequently published art in the form of DE 10 2013 212 276.4. In this connection, it is not a transmission between different coil elements that is measured; instead the coil element acting as an antenna is to be seen as a one-gate sensor element.

A particular configuration provides that at least one coil element integrated in a patient couch of the magnetic resonance device and/or a coil element integrated in a local coil to be arranged posteriorly to the patient is used as a coil element. This has the advantage that the proximity to the patient is established, but no additional components/devices for measuring the breathing are now required. The coil element is already integrated in the patient couch and/or in the posterior local coil, (e.g., to be arranged beneath the patient), which is required anyway. The local coil may be a spinal local coil, for example. In this embodiment the sensor, (e.g., the coil element), moves together with the patient if the patient couch is moved into the patient-receiving mechanism or is moved out of the patient-receiving mechanism, since it is either placed in the local coil that has already been positioned or is placed in the moving part of the examination table, the patient couch, itself. This provides that the breathing data may be acquired as early as immediately after the positioning of the patient outside of the patient-receiving mechanism. A user has the option of checking immediately whether the measurement of the breathing of the patient is working and not just when the patient is already in the patient-receiving mechanism, as would be the case with sensors in the patient-receiving mechanism or the transmission from an anterior coil to a sensor in the patient-receiving mechanism.

A further advantage of this embodiment in which the coil element is integrated in the patient couch or a local coil that is to be positioned beneath the patient is that the spacing between the coil element and the patient is the same, so the value ranges of the characteristics of the reflection properties change only slightly for different types of patient (e.g., slim, obese, muscular). This is in turn in contrast to methods in which a sensor/part of a high-frequency coil arrangement (e.g., body coil) arranged behind the cladding of the patient-receiving mechanism is used.

In a specific embodiment of the method, it may be provided that a signal returning after a transmission signal is separated and measured by a directional coupler. This provides that a transmission signal is output via the coil element that is back-coupled as a reflection signal into the coil element and therefore produces a returning signal that is separated and measured as a measuring signal by a directional coupler. A transceiver unit may in particular be provided therefore that may have a transmitter for transmitting a measuring-transmitting signal, a directional coupler for separating a returning signal, and a receiving device connected downstream of the directional coupler.

The reflection properties are expediently evaluated in the form of the amplitude and/or phase of the returning signal. The value of the amplitude of the returning signal may be considered in the process. As experiments by the applicant have shown, both characteristics clearly indicate the respiratory movement. Both the amplitude and the phase may be evaluated in order to optionally be able to carry out a plausibility check and the like.

The measurement of the reflection properties may be carried out at a frequency outside of the frequency range used for the magnetic resonance imaging. The frequency may specifically be chosen in a range from 0.1-10,000 MHz (megahertz), (e.g., at 20 MHz or at 250 MHz). The measuring frequency for the reflection properties is therefore chosen in such a way that the magnetic resonance imaging is not interfered with. The frequencies are therefore used at which the receiving system of the magnetic resonance device is very insensitive. However, it may be noted in this connection that it is completely conceivable to also use frequencies close to the magnetic resonance frequency (e.g., Larmor frequency), in particular, if the coil element is also used as a receiving coil in the actual magnetic resonance imaging (discussed in more detail below).

As has already been mentioned, the powers used for measurement of the reflection properties may be kept extremely low, so it may be provided by way of example that a transmitting power of the coil element of less than 10 dBm is used for measurement of the reflection properties. No appreciable SAR exposure of the patient needs to be taken into account in this case. There is then no interference, (e.g., saturation), of the sensitive electronic receiving device of neighboring receiving coils, either.

A particularly expedient embodiment provides that, in particular, with coil elements integrated in the patient couch of the magnetic resonance device, at least two coil elements arranged at different positions are used. The coil element used for measurement is chosen on the basis of patient position information that describes the position of the patient relative to the coil elements, in particular, the position of the patient on the patient couch, and/or quality information that describes the quality of the breathing data of the various coil elements. It may therefore be provided that more than a single coil element, (e.g., more than one sensor), is used in order to be able to react flexibly to different positionings of the patient and/or to be more independent of the body size of the patient. Both magnetic resonance examinations in which the patient is positioned in the “head first” orientation and those in which the patient is arranged on the patient couch in the “feet first” orientation, by way of example, are therefore known. If coil elements integrated on different sides of the patient couch are used, the appropriate coil element may be used if, by way of example, the patient position information indicates the appropriate one. The coil element may however also be chosen by way of the analysis of the breathing data quality, which provides in a selection act all coil elements are used for measuring, and the coil element that has the highest signal quality is chosen for the definitive measurement.

The patient position information may include patient data, (e.g., the height, gender, weight, age of the patient, or combinations thereof), and/or be at least partially derived therefrom. Meta information is therefore used to discover which coil element is in the desired position for measuring the breathing of the patient. In addition or alternatively, it may also be provided that the patient position information is determined at least partially from a scout measurement with the magnetic resonance device. Automatic evaluation methods are also already known in this sector that therefore completely automatically determine whether the patient is arranged “head first” or “feet first” on the patient couch and also where the patient is located on the patient couch. It is also possible for the patient position information to be input or for a suitable coil element to even be indirectly selected by an operator.

An embodiment provides that the coil element, (e.g., a coil section of a local coil), is also used for recording magnetic resonance data. If the coil element is also used during magnetic resonance imaging, by way of example since it is integrated in an imaging posterior local coil, then this results in the advantage that no additional breathing sensor antennae are required.

It may firstly be provided in this connection that the coil element is operated in a dual resonant manner at the frequency for a measurement of the reflection properties and at the magnetic resonance frequency. A resonance at the Larmor frequency (magnetic resonance frequency) is then given therefore, but secondly at a breathing sensor frequency at which the measurement of the reflection properties is carried out.

It may also be advantageous, however, if, as an alternative, the measurement of the reflection properties takes place at a frequency adjacent to a noise band for the magnetic resonance imaging and/or a frequency that may be processed by a receiving device for magnetic resonance signals. The frequency for measurement of the reflection properties is therefore chosen in this embodiment close to the magnetic resonance frequency (Larmor frequency), e.g., just outside of the noise band that occurs there, but in such a way that the measurement is not interfered with. This may advantageously enable a receiving channel that exists anyway in the receiving system of the magnetic resonance device, in particular therefore a receiving device there, to be used. Both the signal describing the reflection properties and the measurement of the magnetic resonance signals are then first of all passed to the same receiving device that divides the signals accordingly and feeds them to their respective evaluation devices.

Due to the low powers, an almost continuous measurement of the breathing may be carried out, wherein a cyclical and/or triggered measurement of the reflection properties may also be expedient, however. In this context, it may be provided that a measuring process of the reflection properties is triggered such that the measuring period is outside of a read period and/or an excitation period of a magnetic resonance imaging process. An effect on the magnetic resonance imaging may then largely be ruled out, wherein it may be noted, however, that there is then no breathing data for read time frames of the magnetic resonance frequency and the like. It may therefore be more expedient to skillfully choose the frequency at which the reflection properties are measured such that optimally low influencing of the magnetic resonance imaging occurs.

In this connection, a further advantageous embodiment provides that the coil element has a trap circuit at the magnetic resonance frequency. This provides that measurement of the reflection properties is not affected by the at least at times simultaneously occurring magnetic resonance imaging in this way either, since corresponding signals inside the coil element may be avoided. Conversely, the measurement of the reflection properties may advantageously not interfere with the magnetic resonance imaging either. In the receiving state interference, in other words the resonance, is minimized with the coil elements used in the magnetic resonance imaging. This embodiment is of course most expedient if the coil element is provided so as to be dedicated to the measurement of the reflection properties; it is consequently not used for recording magnetic resonance signals. In a case such as this, the coil element may be used independently of the imaging measures in the magnetic resonance examination.

Apart from the method, the embodiments also relate to a measuring arrangement for measuring the breathing of a patient during a magnetic resonance examination. The measuring arrangement includes at least one coil element positioned beneath the patient (e.g., posteriorly), in the case of a patient positioned for imaging. The measuring arrangement further includes a controller for recording measurement data that describes the reflection properties of the coil element and for determining breathing data that describes the respiratory situation of the patient at various times by evaluation of the measurement data. All statements relating to the method may be transferred analogously to the measuring arrangement, so the controller may expediently be designed to carry out the method. The advantages of the method are also maintained with the measuring arrangement in this way. In certain embodiments, the least one coil element is integrated in the patient couch of a magnetic resonance device and/or a local coil.

The controller may include a transmitter for emitting a measuring-transmitting signal, a directional coupler for separating a returning signal, a receiving device connected downstream of the directional coupler, and an evaluation device. The transmitter, directional coupler, and the receiving device may be combined as a transceiver unit. The evaluation device may be part of an arithmetic device.

The controller may also include a trigger mechanism for triggering measuring processes. Measuring processes may be repeated cyclically by way of example for the reflection properties, although it is also conceivable to make these dependent on other events that occur, by way of example certain events during the course of the magnetic resonance examination.

Finally, the embodiments also relate to a magnetic resonance device having a measuring arrangement. All statements relating to the measuring arrangement and method may be transferred analogously to the magnetic resonance device in which the at least one coil element is integrated in the patient couch and/or a local coil.

### BRIEF DESCRIPTION OF THE DRAWINGS

FIG. 1 depicts a schematic diagram of an example of a magnetic resonance device.

FIG. 2 depicts an example of components of a measuring arrangement.

FIG. 3 depicts an example arrangement of a coil element relative to a patient.

FIG. 4 depicts an example of a measured amplitude of a returning signal in the case of a first patient.

FIG. 5 depicts an example of a measured phase of the returning signal in the case of the first patient.

FIG. 6 depicts an example of a measured amplitude of the returning signal in the case of a second patient.

FIG. 7 depicts an example of a measured phase of the returning signal in the case of the second patient,

FIG. 8 depicts an example of a coil element integrated in a local coil.

FIG. 9 depicts an example of a patient couch having a plurality of coil elements.

### DETAILED DESCRIPTION

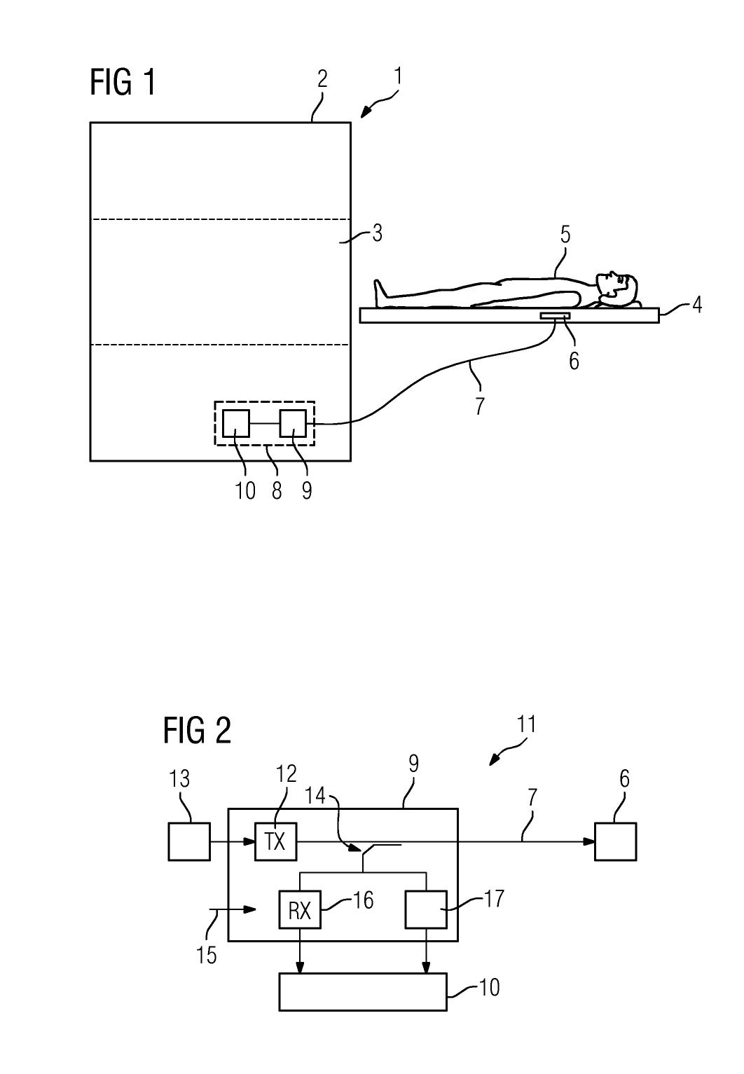

FIG. 1 depicts a schematic diagram of a magnetic resonance device 1. The magnetic resonance device has a main magnetic unit 2 that, here indicated in broken lines, defines a cylindrical patient-receiving mechanism 3. A high-frequency coil arrangement (e.g., body coil) and a gradient coil arrangement may be provided so as to surround the patient-receiving mechanism 3. The main magnet generating the basic magnetic field is likewise provided in the main magnetic unit 2.

By a patient couch 4, a patient 5 positioned on the patient couch 4 may be moved into the patient-receiving mechanism 3 to carry out a magnetic resonance examination, e.g., to conduct magnetic resonance imaging. If this magnetic resonance imaging takes place in the region of the abdomen and/or thorax of the patient 5, then this region is subject to the respiratory movement of the patient 5. Therefore, the breathing of the patient 5 may also be measured in the present case, for which purpose the magnetic resonance device 1 has a corresponding measuring arrangement. This includes a coil element 6 integrated in the patient couch 4, which is therefore arranged posteriorly from the patient in the region of his abdomen and/or thorax. Due to the positioning inside the patient couch 4, the coil element 6 is also arranged very close to the back of the patient 5, e.g., less than 5 cm away from the back of the patient 5. The coil element 6 is fixed in position in relation to the patient 5, so measurements, (e.g., for checking the operation), may also be carried out even in the case of a patient 5 located outside of the patient-receiving mechanism 3. The coil element 6 is connected to a controller 8 (depicted only schematically) of the magnetic resonance device 1 by a cable connection 7, here a coaxial cable made of copper. The controller 8, in addition to operation of the magnetic resonance device 1 itself, also controls the operation of the measuring arrangement according to the method and acts as a controller of the measuring arrangement therefore. For this purpose, the controller 8 includes in particular a transceiver unit 9 and an evaluation device 10.

This is explained in more detail by the schematic diagram of the measuring arrangement 11 in FIG. 2. The transceiver unit 9 accordingly includes a transmitter 12 in which a measuring-transmitting signal is produced that is output via the coil element 6. This may occur cyclically, for which purpose the transmitter 12 receives the signal from a clock 13.

If the measuring-transmitting signal is irradiated via the coil element 6 into the body of the patient 5, then the signal is affected by the properties of the tissue, whose position is changed by the breathing, however, so different returning signals respectively are produced as a function of the respiratory situation. These returning signals describe the reflection properties. They are separated by a directional coupler 14 and fed to a, in this case, two-part receiving device 15, in which: (1) the amplitude of the returning signal is digitized in an amplitude unit 16, and (2) the phase shift that has occurred is determined in a phase unit 17 by way of comparison with the transmission signal and is likewise digitized. This measurement data, e.g., the amplitude and the phase of the returning signal, is forwarded to the evaluation device 10. These evaluate the amplitude characteristic and the phase characteristic to produce the breathing data that describes the breathing phases at various times, and this may be used by the controller 8, by way of example, for triggering magnetic resonance measurements and/or for retrospective gating.

It may also be noted at this point that even low transmitting powers are sufficient to obtain a meaningful returning signal, which may also be called a breathing signal.

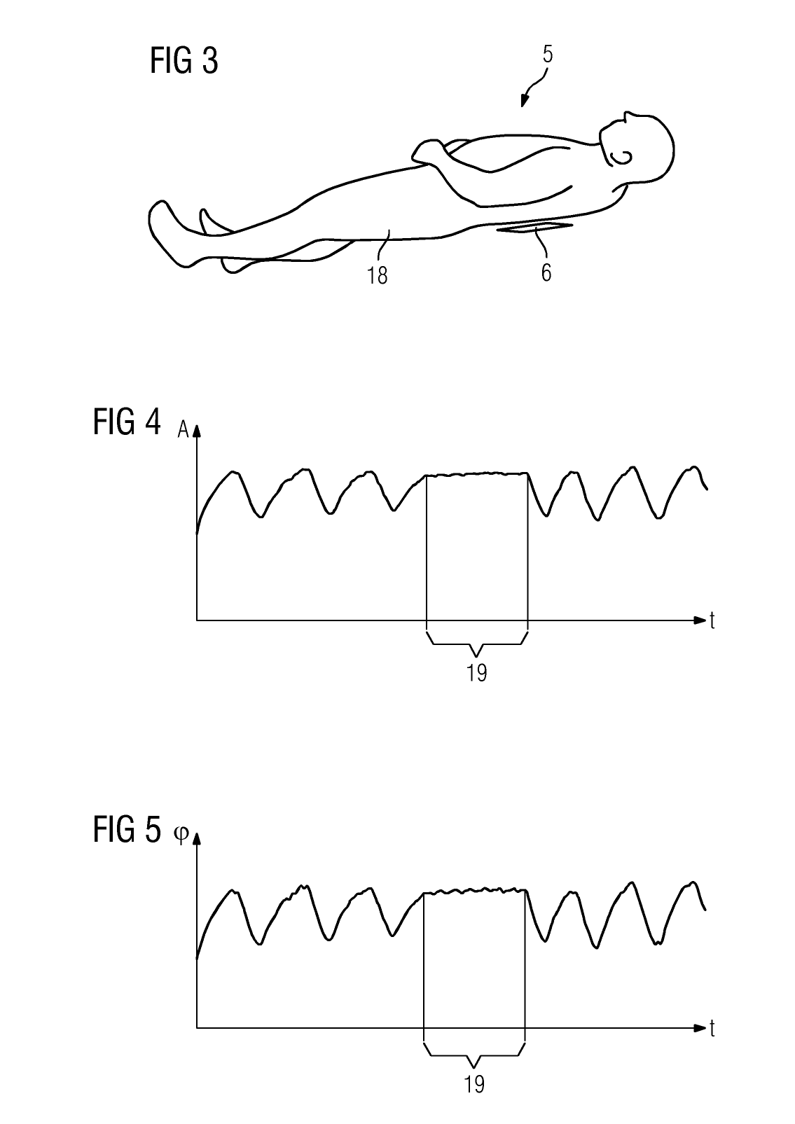

FIG. 3 depicts for the purpose of clarification a perspective view for the arrangement of the coil element 6 relative to the body 18 of the patient 5. As may be seen, the coil element 6 is arranged beneath, e.g., posteriorly to the body 18, such as in the middle region of the back.

The coil element 6 may be used for recording magnetic resonance data during the course of the magnetic resonance imaging. The coil element 6 may be designed so as to be dual resonant in order to implement transmitting and receiving at the magnetic resonance frequency and at the frequency for the measurement of the reflection properties. It is also possible to carry out the measurement of the reflection properties at a frequency that directly adjoins a noise range of the frequency range used for magnetic resonance imaging and may therefore optionally be evaluated via the same receiving channel without a dual resonance being necessary.

In certain embodiments, the coil element 6 is not used for magnetic resonance imaging and may therefore be operated independently of the magnetic resonance imaging at a frequency that is outside of the frequency range for the magnetic resonance imaging. Measurements using a coil element of this kind are depicted, by way of example, by FIGS. 4 to 7. FIGS. 4 and 5 relate to a first patient, wherein FIG. 4 depicts the course over time of the amplitude of the returning signal and FIG. 5 depicts the course over time of the phase of the returning signal. The measurement was carried out at a frequency of 20 MHz. The breathing cycles and the holding of breath in a period 19 may clearly be seen.

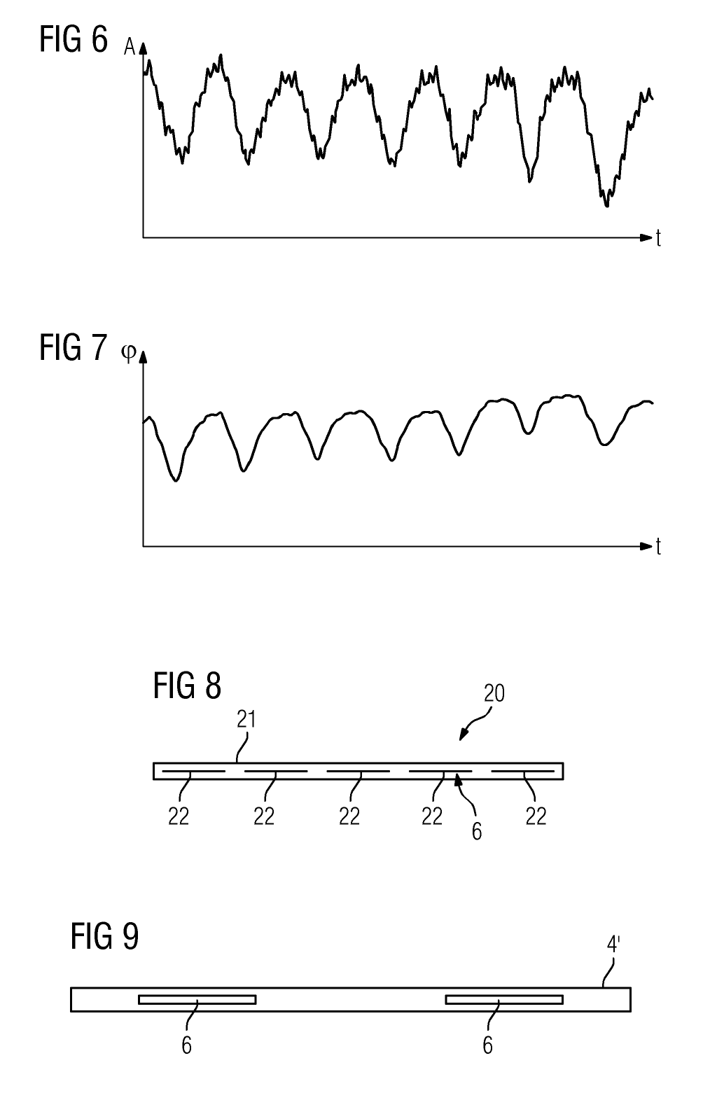

FIGS. 6 and 7 depict further measuring results for a second patient, wherein FIG. 6 relates to (the value of the) amplitude and FIG. 7 relates to the phase. There is no holding of breath here. Since the second patient is a very slim woman, even the heartbeat may be seen superimposed on the breathing signal. Similar measuring results to those in FIGS. 4 to 7 were also obtained at frequencies of 250 MHz.

FIG. 8 depicts a schematic diagram of a local coil 20, here a spinal coil, which may be placed under the patient 5 on the patient couch 4. Various coil sections 22 are embedded in a flexible carrier material 21. One of these coil sections that is advantageously positioned may likewise be used as a coil element 6, so the coil element 6 is not integrated in the patient couch 4 but in the local coil 20.

FIG. 9 depicts a modified embodiment of a patient couch 4′, in which two coil elements 6 for measuring the breathing of a patient are integrated symmetrically on opposing sides. The breathing of the patient 5 may be measured in every case in this way irrespective of whether the patient is positioned “head first” or “feet first” on the patient couch 4′. If the head of the patient 5 is on the right-hand side of the patient couch 4′, then the right-hand coil element 6 will also deliver better results for the breathing data, otherwise the left-hand coil element 6 will do this. Therefore, a coil element 6 is chosen that may deliver the measuring results. This may be determined as a function of patient position information that may be determined from a scout measurement and/or, in particular, in the case of a relatively large number of coil elements 6, patient data, (e.g., height, weight, and the like). Inputting patient position information is of course also possible, as is the direct choice of a coil element 6 to be used. The choice of coil element 6 may also be made as a function of quality information, for the determination of which test measurements are carried out using all coil elements 6 in the case of the positioned patient 5 and the coil element 6 that delivers the highest quality measurement data or breathing data is used. This exercising of choice may also take place automatically via the controller 8.

In certain embodiments, more than two coil elements may be used.

It may also be mentioned in this context that the measuring methods proposed here may also be used in addition to an existing method, such as the use of a breathing cushion or breathing belt. Even in a case such as this, the operator and/or controller 8 may decide that measuring method is used in the specific case. As a setting, a measurement may take place such as via the coil element 6, it being possible to evaluate this with higher priority only if a further sensor, (e.g., a breathing cushion), has been connected.

Reference is made to the fact that, in particular, with coil elements 6 that are not used for the measurement of magnetic resonance signals, a trap circuit may expediently be part of the coil element for the magnetic resonance sequence in order to avoid or minimize interference to the breathing measurement due to the magnetic resonance imaging and vice versa.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for measuring the breathing of a patient during a magnetic resonance examination with a magnetic resonance device, the method comprising:
measuring and evaluating reflection properties of at least one coil element arranged beneath the patient to determine, at various times, breathing data that describes a respiratory situation of the patient.

2. The method as claimed in claim 1, wherein the at least one coil element is integrated (1) in an patient couch of the magnetic resonance device, (2) in a local coil configured to be arranged posteriorly to the patient, or (3) in the patient couch and in the local coil.

3. The method as claimed in claim 1, wherein a signal that returns following a transmission signal is separated and measured by a directional coupler.

4. The method as claimed in claim 3, wherein the reflection properties are evaluated in the form of an amplitude, a phase, or the amplitude and the phase of the returning signal.

5. The method as claimed in claim 1, wherein measurement of the reflection properties is carried out at a frequency that is outside of a frequency range used for magnetic resonance imaging.

6. The method as claimed in claim 5, wherein the frequency is in a range from 0.1 to 10,000 MHz.

7. The method as claimed in claim 1, wherein a transmitting power of the coil element of less than 10 dBm is used for measurement of the reflection properties.

8. The method as claimed in claim 1, wherein at least two coil elements are integrated at different positions in the patient couch of the magnetic resonance device, wherein the coil elements used for measurement are chosen based on patient position information that describes a position of the patient relative to the coil elements, quality information that describes a quality of the breathing data of the coil elements, or both the patient position information and the quality information.

9. The method as claimed in claim 8, wherein the patient position information comprises or is derived at least partially from patient data comprising height of the patient, gender of the patient, weight of the patient, age of the patient, or combinations thereof.

10. The method as claimed in claim 8, wherein the patient position information is determined at least partially from a scout measurement with the magnetic resonance device.

11. The method as claimed in claim 1, wherein a coil section of a local coil is used for recording magnetic resonance data.

12. The method as claimed in claim 11, wherein the coil element is configured to be dual resonant in the case of a frequency for the measurement of the reflection properties and in the case of the magnetic resonance frequency.

13. The method as claimed in claim 11, wherein the measurement of the reflection properties occurs at a frequency adjacent to a noise band for the magnetic resonance imaging and/or a frequency that is processed by a receiving device for magnetic resonance signals.

14. The method as claimed in claim 1, wherein the coil element is spaced apart from a body of the patient by less than 10 cm.

15. The method as claimed in claim 1, wherein a measuring process of the reflection properties is triggered such that a measuring period lies outside of a read period, an excitation period, or the read period and the excitation period of the magnetic resonance examination.

16. A measuring arrangement for measuring breathing of a patient during a magnetic resonance examination with a magnetic resonance device, the measuring arrangement comprising:
at least one coil element positioned beneath the patient positioned for imaging; and
a controller configured to record measurement data that describes reflection properties of the coil element and to determine breathing data that describes a respiratory situation of the patient at various times by evaluation of the measurement data.

17. The measuring arrangement as claimed in claim 16, wherein the controller comprises a transmitter for transmitting a measuring-transmitting signal, a directional coupler for separating a returning signal, a receiving device connected downstream of the directional coupler, and an evaluation device.

18. The measuring arrangement as claimed in claim 17, wherein the controller further comprises a trigger mechanism for triggering measuring processes.

19. The measuring arrangement as claimed in claim 16, further comprising an additional coil element that is not used in the imaging, the additional coil element comprising a trap circuit at a magnetic resonance frequency.

20. A magnetic resonance device comprising:
a measuring arrangement comprising:
at least one coil element positioned beneath a patient positioned for imaging; and
a controller configured to record measurement data that describes reflection properties of the coil element and to determine breathing data that describes a respiratory situation of the patient at various times by evaluation of the measurement data.
